(19) United States
(12) Patent Application Publication
(10) Pub. No.: US 2010/0094316 A1
(43) Pub. Date: Apr. 15, 2010

(54) METHOD AND DEVICE FOR FIXING AND/OR MANIPULATING TISSUE

(76) Inventors: Heinz Rupp, Marburg (DE); Bernhard Maisch, Marburg (DE); Thomas Philipp Rupp, Marburg (DE); Ekkehard Schüler, Marburg (DE); Karin Rupp, Marburg (DE)

Correspondence Address: CLARK & BRODY, 1090 VERMONT AVENUE, NW, SUITE 250, WASHINGTON, DC, 20005, US

(21) Appl. No.: 12/448,110

(22) PCT Filed: Dec. 10, 2007
(86) PCT No.: PCT/EP2007/010740

§ 371 Date: Dec. 14, 2009

(30) Foreign Application Priority Data
Dec. 10, 2006 (DE) 10-2006-058-447.3

(51) Int. Cl. A61B 17/08

(52) U.S. Cl. 606/151

(57) ABSTRACT

The invention concerns a device for the fixation and manipulation of those tissues which are at least temporarily suitable for being fixed by suction.

Hereby, tissues are understood to be, in addition to organic tissues, such as e.g. tissues of animals and humans, also inorganic tissues, such as films or textiles.

In particular, the invention concerns a device for the fixation and manipulation of tissues which surround sensitive organs, such as e.g. the pericardium in humans.

Furthermore, the invention concerns a method for the fixation and manipulation and, in particular, for puncturing of such tissues or the underlying areas.

The invention concerns a device for the fixation and/or manipulation of tissues according to the generic term of claim 1 as well as a method for the fixation and/or manipulation of tissues according to the generic term of claim 21.

Devices for the fixation and/or manipulation of tissues are already known. Thus, U.S. Pat. No. 5,827,216 describes, for example, a device for the fixation, lifting and puncturing of the pericardium with a body which penetrates the tissue of the pericardium and/or the underlying areas. This is preferentially realized by a hollow needle which is arranged in a longitudinally moveable manner within a guiding body and is able to be inserted into the interior of a suction head for the tissue. The suction head is arranged on the front (distal) end of the guiding body and provided with a side opening through which the interior is accessible from outside. A vacuum which passes through the guiding body is able to be generated by connecting a suction device to the entire device. When the suction head is placed with its side opening on the pericardium, the latter is absorbed through the opening into the interior. A bubble is formed which is subsequently able to be punctured with the needle. Liquids can be drawn off or pharmaceuticals can be introduced through the hollow needle. A wire is also able to be inserted into the pericardial sac through the hollow needle.

Examinations of different tissues have shown that sufficient fixation of the pericardium and access to the pericardial sac have only been successful when the absorbed tissue was sufficiently thin. However, if the tissue is thickened, for instance as a consequence of adiposis, inflammation or scarring, the interior of the suction head is only filled with tissue. No bubble is formed which enables access to the pericardial sac. The needle introduced through the guiding body punctures only into the tissue filling the interior and does not reach the underlying pericardium area. Furthermore, a thickened pericardium can congest the opening so much that only a little or no tissue at all reaches the interior. Thus, reliable fixation of the tissue, as well as a puncture with the needle or another penetrating body, are impossible.

A further disadvantage of the mentioned devices lies in the fact that the tissue cannot be stretched sufficiently within the suction head. In this case, the pericardium would give way when attempting to puncture it. A penetrating body, e.g. a flexible, in most cases blunt wire would deflect and not perforate the pericardium (tent).

U.S. Pat. No. 5,972,013 discloses a device which is also supposed to allow minimally invasive access to the pericardium of the heart of a human or an animal. The needle is guided within a guide tube which provides a frontal opening on its distal end to draw in the pericardium. By introducing the needle into the interior after the suction of the tissue, the end of the needle is deflected laterally to the direction of movement, in such a way that the needle penetrates the pericardium in an inclined manner.

Here, it is also a disadvantage that thickened tissue cannot be drawn at all, or only in an insufficient manner, into the suction head, so that puncturing with the penetrating body can not occur at all or only within the tissue itself. Furthermore, it is problematic that manipulation with the needle is primarily realized perpendicularly to the surface of the tissue and, thus, perpendicularly to the mostly beating heart located below. Unintentional puncture of the heart muscle is, however, a life-threatening injury which can even lead to death of the patient within a very short time.

It is the aim of the present invention to overcome these and other disadvantages of the state of the art and to provide a device which enables fixation and/or manipulation of a tissue, which is always reliable, even when the latter is present in a thickened state. A penetrating body guided within the device has to puncture the tissue rapidly and precisely and to optimally attain an area located below the tissue. Furthermore, the device has to be assembled at a reasonable price and easily manageable.

A further aim of the invention is to provide a method to always fix and/or manipulate a tissue in a secure manner, even when the tissue is present in a thickened state. The latter has to be able to be stretched sufficiently in order to enable a penetrating body to penetrate and find access to an area located below the tissue.

The main characteristics of the invention are indicated in claims 1, 21, 24, 25 and 26. Embodiments are the subject matter of claims 2 to 20 and 22 to 23.

In the case of a device for fixation and/or manipulation of tissue and/or areas located below, with at least one body penetrating the tissue and/or the areas located below and one guiding body for the penetrating body, wherein the guiding body comprises means to guide the penetrating body, wherein the guiding body has or forms a suction head at its end, wherein the guiding body comprises within the suction head an interior with at least one opening arranged in the side area to draw in the tissue to be punctured and/or manipulated and wherein the guiding body comprises means for the introduction of a vacuum into the interior, the invention provides that at least one means for mechanical fixation and/or attachment of the tissue to the suction head is provided in the area of the opening of the suction head.

Through the means for mechanical fixation and/or attachment of the tissue to the suction head, significantly stronger and more reliable attachment of a tissue has been achieved as compared to traditional devices. The tissue is not only drawn in by the suction head but also mechanically fixed, enabling significantly improved handling of the entire device, in particular of the penetrating body. Hereby, the device according to the present invention can be used for every tissue able to be absorbed, in particular for human or animal tissue of any thickness, especially in the context of minimally invasive surgery where there is no direct visual control and where it has to be ensured that tissue for subsequent manipulation is attached to a special device, e.g. to a device for puncturing and/or addition/removal of materials into and/or out of the area below the tissue.

The solution according to the present invention opens completely new handling possibilities of the device, in particular and especially during lifting of a possibly thickened pericardium, as additional mechanical fixation of the tissue to the suction head takes place in the new device, followed by manipulation, e.g. the forward movement of a wire, a dilator or a needle, preferentially perpendicular to the clamping, into the tissue with variable thickness. Through the clamping, the tissue is no longer able to avoid the manipulation and even a flexible wire always results in a reliable and precise perforation of the tissue. In the case of a flexible wire, additional guidance for the wire can be provided within the device in such a way that it is not able to avoid the opposing force during piercing of the tissue. The advantage of this device with flexible wire is that the latter is no longer guided within the pericardial sac after piercing the pericardium and, thus, is unable to injure the opposing epicardium. In fact, the guide wire will freely move within the pericardial sac. After moving the instrument back, the guiding wire is able to remain within the pericardial sac and will be used, e.g., to move a dilator forward.

One embodiment of the invention provides that at least one means for mechanical fixation and/or attachment of the tissue to the suction head is elastic stretching or clamping elements. These ensure that the tissue always finds a reliable hold to the suction head. Hereby, each elastic stretching or clamping element can hereby form an elastic lip, spring or tongue which protrudes at least partially into the opening. Alternatively, the elastic stretching or clamping element is able to form a surrounding elastic edge for the opening.

Through this embodiment, the elastic stretching or clamping elements are initially separated from one another by the tissue when the latter is drawn in the interior of the suction head. They recede, e.g. into the interior and grasp the tissue, as soon as the suction force declines and an external force interacts with the tissue.

Hereby, it is an advantage if the cross-section of the opening which is limited by the elastic stretching or clamping elements is smaller than the cross-section of the interior limited by the opening. Therefore, the cross-section of the opening which is limited by the elastic stretching or clamping elements is expanded during the absorption of the tissue. If one subsequently pulls on the tissue, the opening is reduced. The tissue is fixed to the suction head; the stretching or clamping elements generate a retention force which additionally fixes the tissue to the suction head. This enables much better fixation of the tissue. The device is able to be turned to lift, for example, relatively thick tissue in order to, e.g., lift an outer or upper tissue from an interior tissue or area.

The elastic stretching or clamping elements are able to be actively moveable or modifiable, wherein a mechanical, electrical, magnetic, hydraulic or pneumatic drive is able to be used.

Complementary or alternatively to the stretching or clamping elements, the at least one means for the mechanical fixation and/or attachment of the tissue to the suction head is able to be the penetrating body itself, by inserting the latter, for example, to a certain extent into the already absorbed tissue. This also results in reliable mechanical fixation of the tissue to the suction head.

For that purpose, it is appropriate that the penetrating body is slide guided outside the suction head. Therefore, the manipulation itself also takes place outside the suction head. In the example of accessing the pericardium, the suction head is able to be turned approx. 90 degrees to the right, whereby the attached tissue is lifted upwards. After the turning, the penetrating body (e.g. a needle) is inserted into the resulting tissue fold or the resulting “tent”; the space below is able to be reached optimally. Such a procedure is suitable for being carried out with a probable multitude of patients whose pericardium thickness is of 2 to 3 mm.

Concerning the construction, it is advantageous if the means for guidance of the penetrating body are realized separately from the means for the introduction of a vacuum into the interior. This does not only simply the construction of the device. The penetrating body does not have to be sealed against the vacuum.

The penetrating body is able to be—depending on the application purpose—a needle, an electrode, a wire, a loop, a cutting device or something similar.

An important further development of the invention provides that the means for the introduction of a vacuum into the interior is a vacuum channel which ends in the interior with a vacuum opening, wherein the vacuum opening is realized at a distance from the edge of the opening. As a result, the vacuum opening is not covered that easily during absorption of the tissue into the interior of the suction head. Significantly more tissue or a significantly larger tissue area can be absorbed into the interior, before the suction effect reduces due to the covering of the channel conducting the vacuum.

In morbidly obese patients, fat accumulation does not occur exclusively in the abdomen, but also on the pericardium in the form of fat deposition. In those patients, the tissue fold resulting from turning the device consists of fat tissue and the needle pushed forward no longer reaches the pericardial sac. In this case, it also serves for the mechanical fixation of the mostly heavy tissue.

In order to be able to carry out nevertheless reliable manipulation, the invention provides that at least one further guidance for another penetrating body is provided in the suction head. This further guidance is preferably realized in a rotatable manner in relation to the suction head.

With this device, access to the pericardial sac is possible even when the pericardium is formed in an extremely thick manner, for example, through local fat depositions or scarring. Thus, the instrument provided for clinical utilization is not only suitable for being used with normal, but also with overweight patients.

The further penetrating body is able to be a guide wire, a needle, an endoscope or something similar.

A further different embodiment of the invention provides that the suction head comprises at least one molding. This can be, e.g., a support strip which is realized in the border area of the suction head.

The suction head is able to support itself on the molding during its rotation in such a way that the tissue fixed to the suction head is lifted higher from its subsurface. The resulting gap, i.e. the area located below the tissue, is better accessible.

It is appropriate to realize the suction head in the area of the side opening in a flattened manner. Thus, the device is suitable for being placed evenly on the tissue.

On the suction head of the device, a cutting device for the tissue is able to be realized. Thereby, further manipulations are possible.

The method according to the present invention for the fixation and/or manipulation of tissue and/or areas located below comprises the following steps:
a) approach of the device according to the present invention to the tissue to be fixed;
b) suction of the tissue through the side opening of the device until tissue is attached and mechanically fixed;
c) further lifting or raising of the tissue from its position before step b)
d) approach of the at least one penetrating body to the tissue in the spaced area between the position of the tissue before step b) and the tissue area in the interior of the device spaced the furthest therefrom.

Thus, a more reliable method for the fixation and subsequent manipulation is achieved; in particular also for such cases—as described above—in which the tissue is very thick-walled and manipulation in the interior of the device does not allow accessibility to the areas located below.

A particularly advantageous first embodiment of the method results from a variation of step c) in a step c2) (claim 22), wherein then the further lifting or raising takes place through rotation of the oblong hollow body around its longitudinal axis and, thus, also of the tissue attached to the hollow body in the area of the side opening. Through this step, the manipulation area is rotated away from the sensitive areas located beneath the tissue (e.g. the heart muscle) and, thus, protected during the subsequent manipulation.

An advantageous, second embodiment of the method provides the variation of step d) with the variant d2) (claim 23), which provides the approach of manipulation elements to the tissue in the spaced area between the position of the tissue before step b) and the tissue area in the interior of the device spaced the furthest therefrom outside of the area of the interior of the hollow body conducting the vacuum. Through this, manipulation can also take place outside of the interior of the hollow body in the area of the side opening, which allows more reliable accessibility to the area beneath the tissue, particularly in thick-walled tissues.

The combination of the first embodiment of the method with a further embodiment of the device, in which a prominence in the form of a ridge or wall (also called a ground lip in the following) is mounted in the area of the side opening on the outer part of the guiding body or of the hollow body of the device, is particularly advantageous. Through this, on one hand the friction between tissue and the device is increased, and, furthermore, this ground lip serves to form a barrier between the area of manipulation and the sensitive area located beneath the raised tissue after a corresponding rotation, as in step c2).

In the case of a suction head for the absorption of tissue, comprising a body with an interior, which is realized for the addition of a vacuum to the interior, and wherein the body comprises an opening on the surface, which is connected to the interior for the addition of vacuum to the opening, the invention provides that the opening is molded or arranged to be molded in such a way that the cross-section of the opening, for instance through a surrounding edge or individual pieces protruding into the plane of the opening or in another form, is realized smaller than the cross-section of the interior of the suction head located below, parallel to the plane of the opening.

Further characteristics, details and advantages of the invention result from the text of the claims, as well as from the following description of embodiments through the figures. They show:

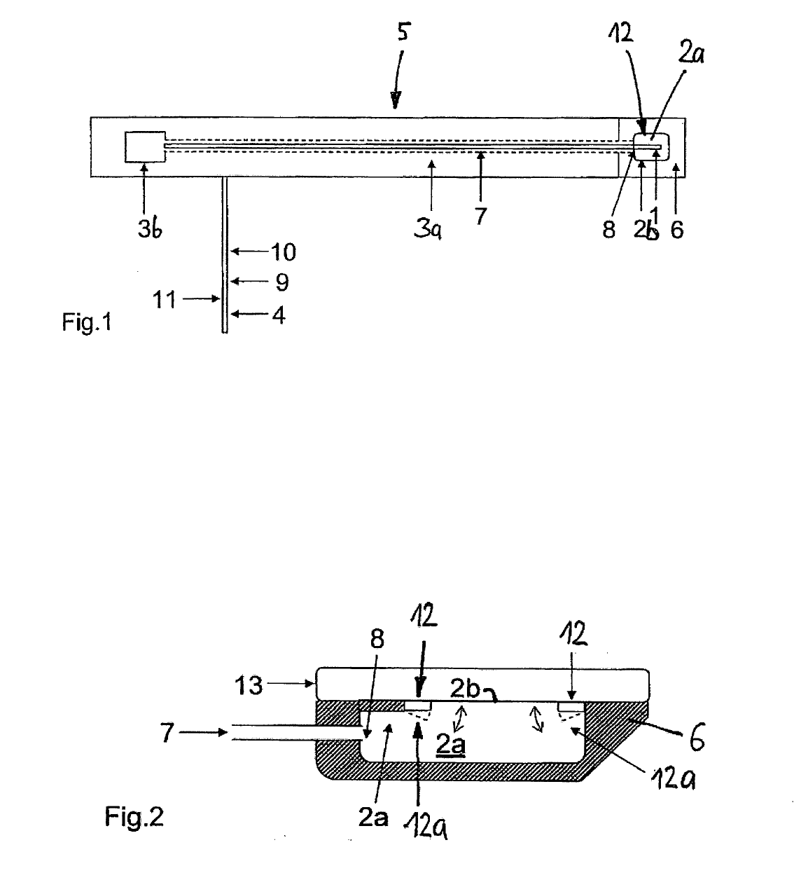

FIG. 1 shows a view from above of the device according to the present invention;

FIG. 2 a sectional drawing approximately in the longitudinal plane through the section head of the device;

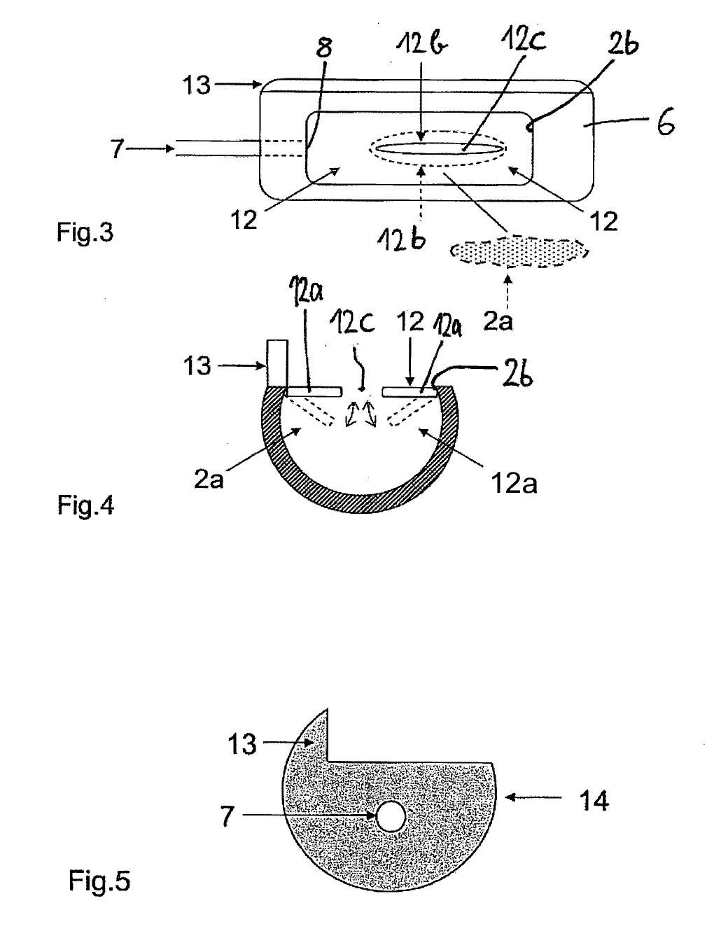

FIG. 3 a view from above of another embodiment of a suction head of the device;

FIG. 4 a sectional drawing approximately perpendicular to the longitudinal plane through the section head of FIG. 2;

FIG. 5 a sectional drawing located approximately perpendicular to the longitudinal plane through a tool holder for guiding the suction head;

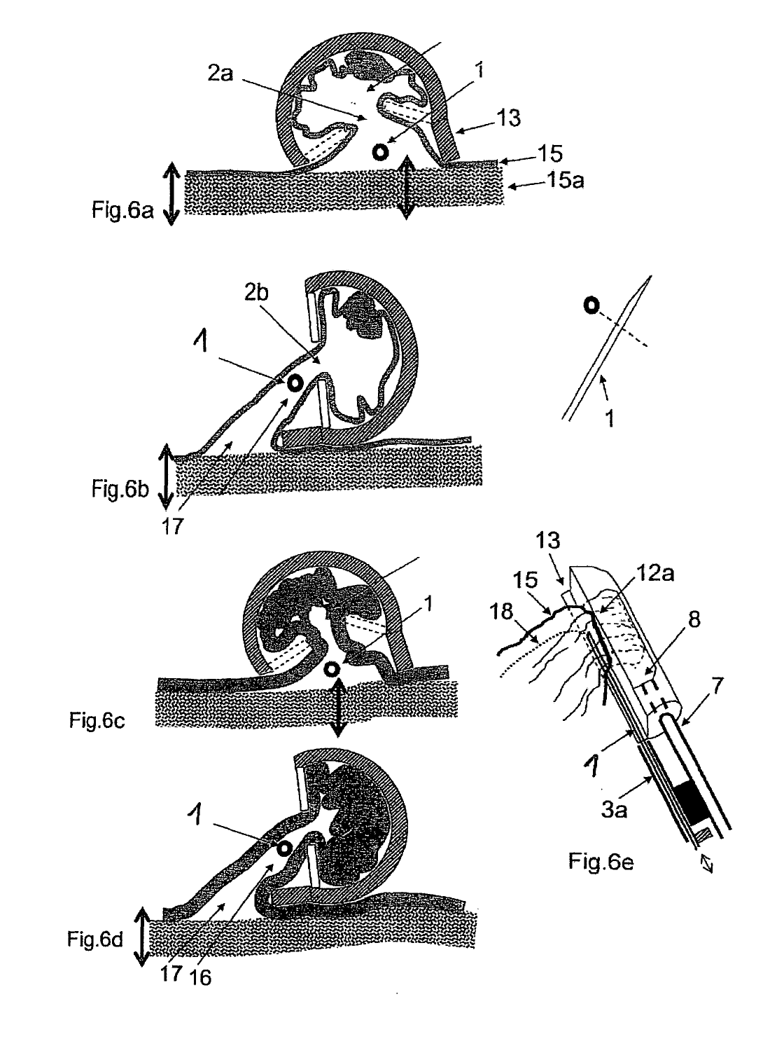

FIG. 6a a suction head with absorbed tissue after step b) of the method according to the present invention;

FIG. 6b the suction head after rotation to the right of about 90° after step c2) of the method according to the present invention;

FIG. 6c,d the suction head with absorbed tissue with a particularly thick wall;

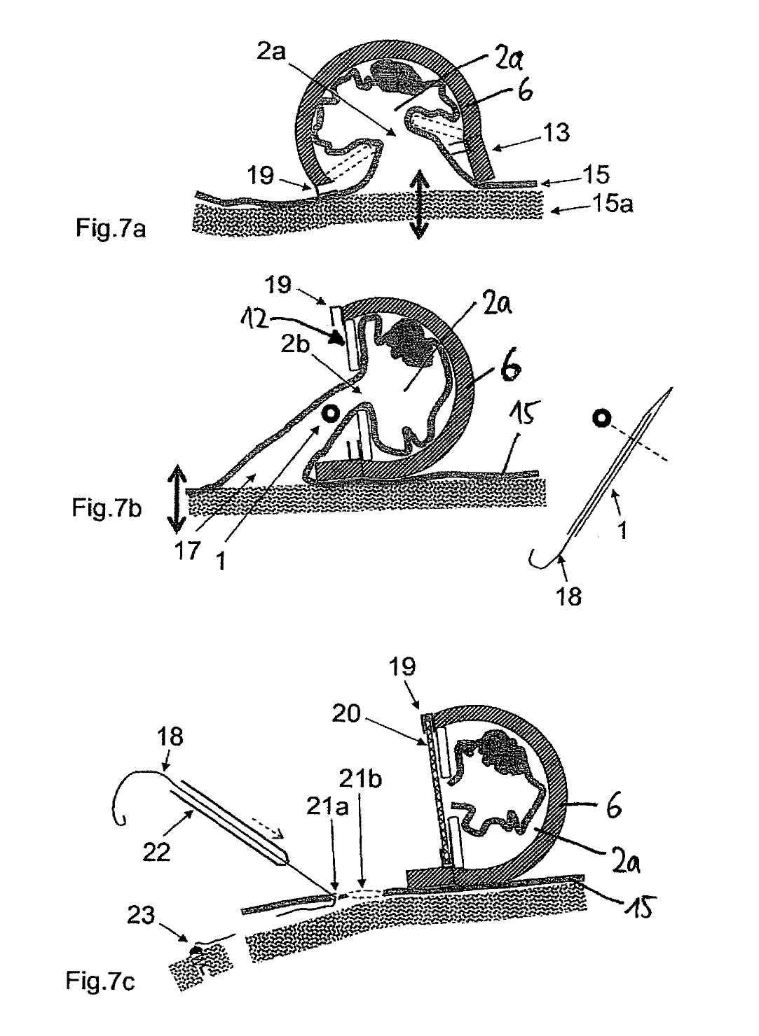

FIG. 7a an embodiment of the device with a guidance for a cutting device located in the suction head, wherein tissue is being absorbed into the suction head;

FIG. 7b the suction head after rotation of 90° (method step c2);

FIG. 7c the suction head after separation of the absorbed tissue;

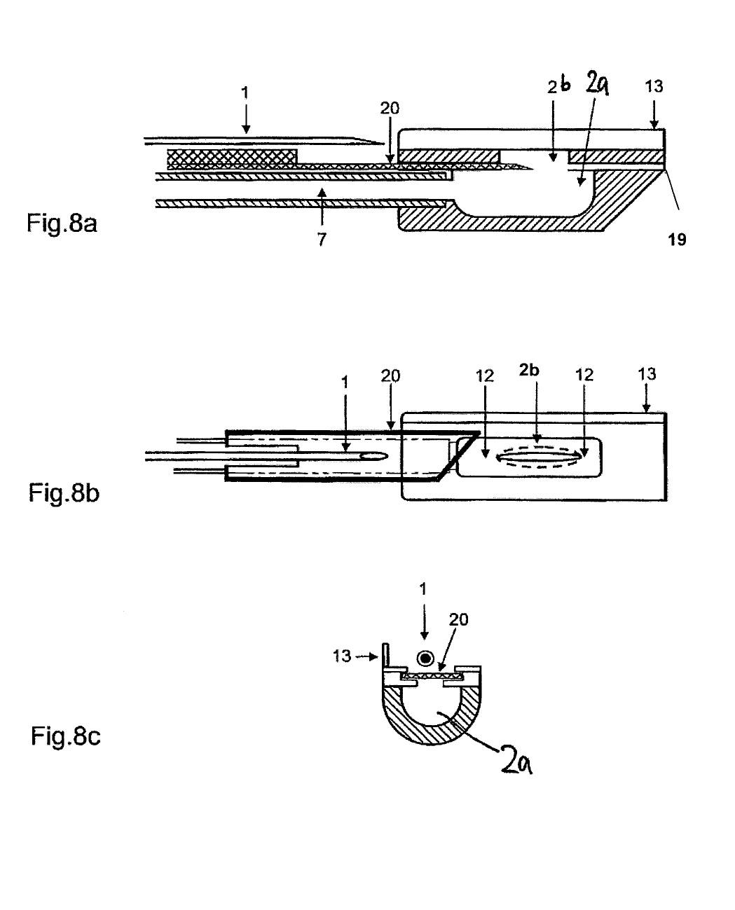

FIG. 8a a sectional drawing approximately in the longitudinal plane of FIG. 7;

FIG. 8b a view from above of the suction head of FIG. 7;

FIG. 8c a sectional drawing approximately in the longitudinal plane of the suction head of FIG. 7;

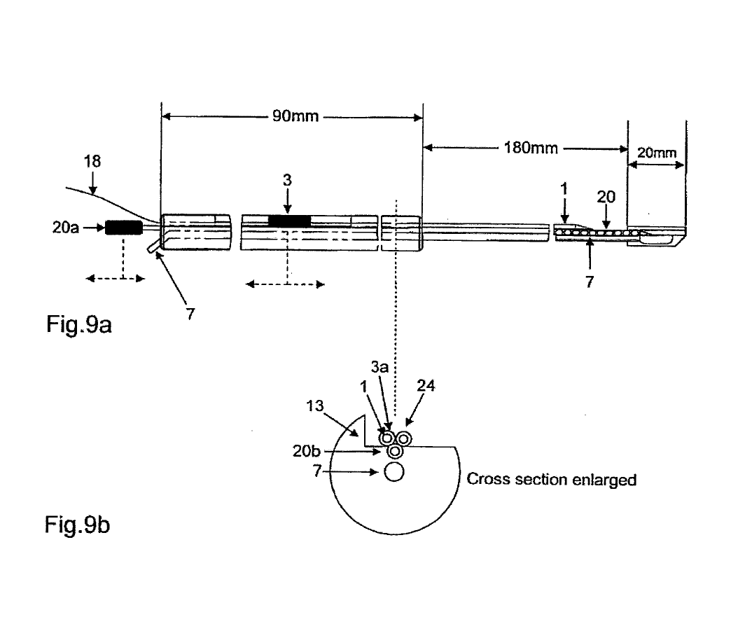

FIG. 9a,b a further embodiment of a device according to the present invention;

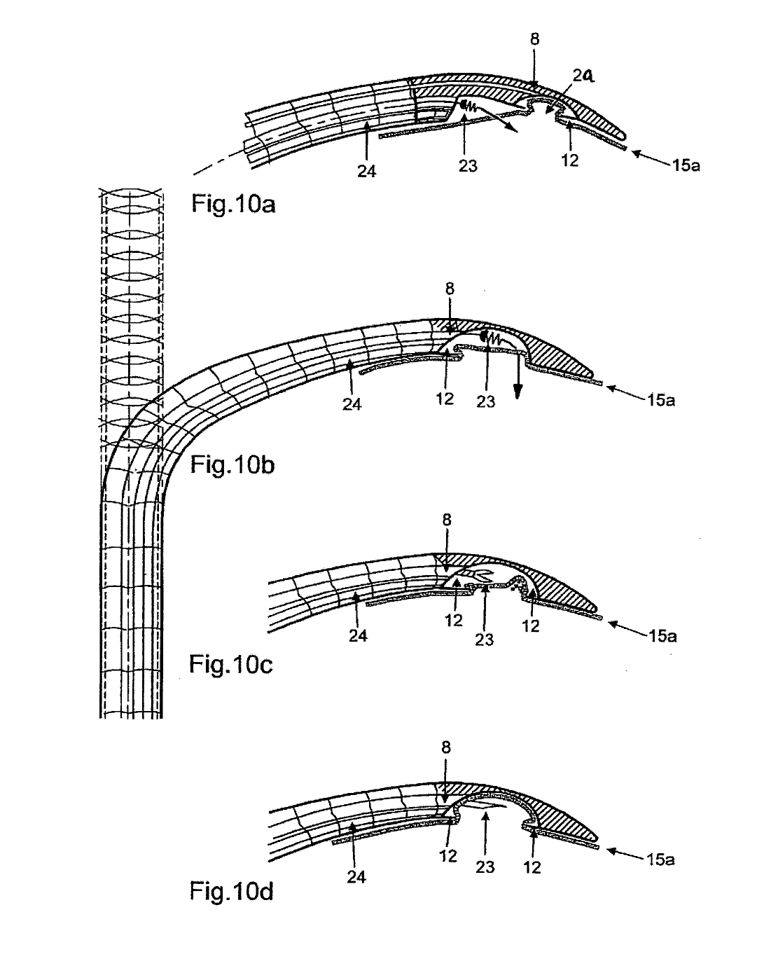

FIG. 10 a flexible embodiment of the hollow or guiding body with different suction heads and manipulation devices;

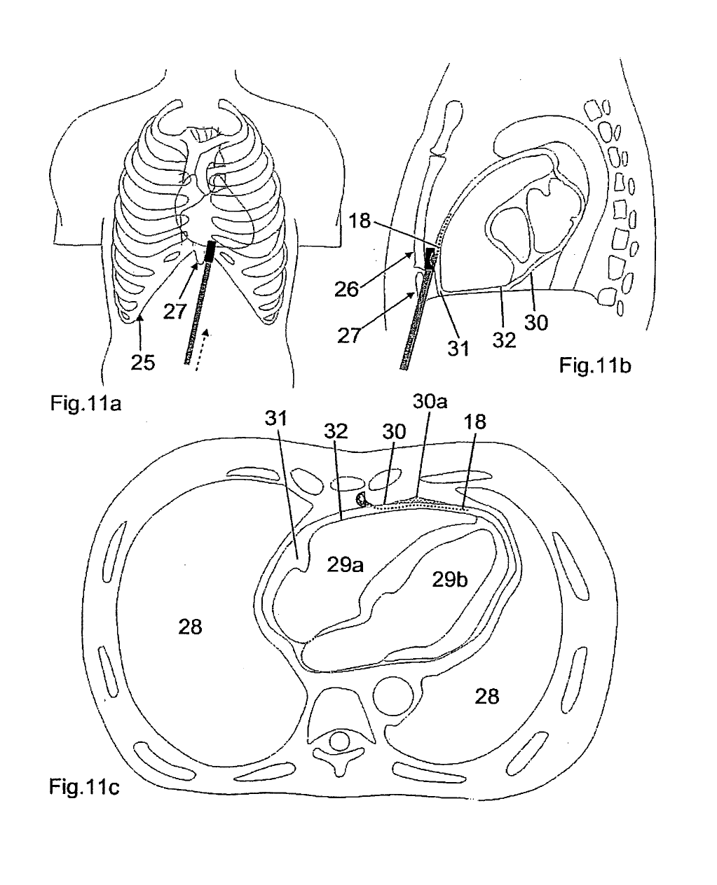

FIG. 11 a schematic of the procedure in the case of minimally invasive manipulation in or at the pericardial sac;

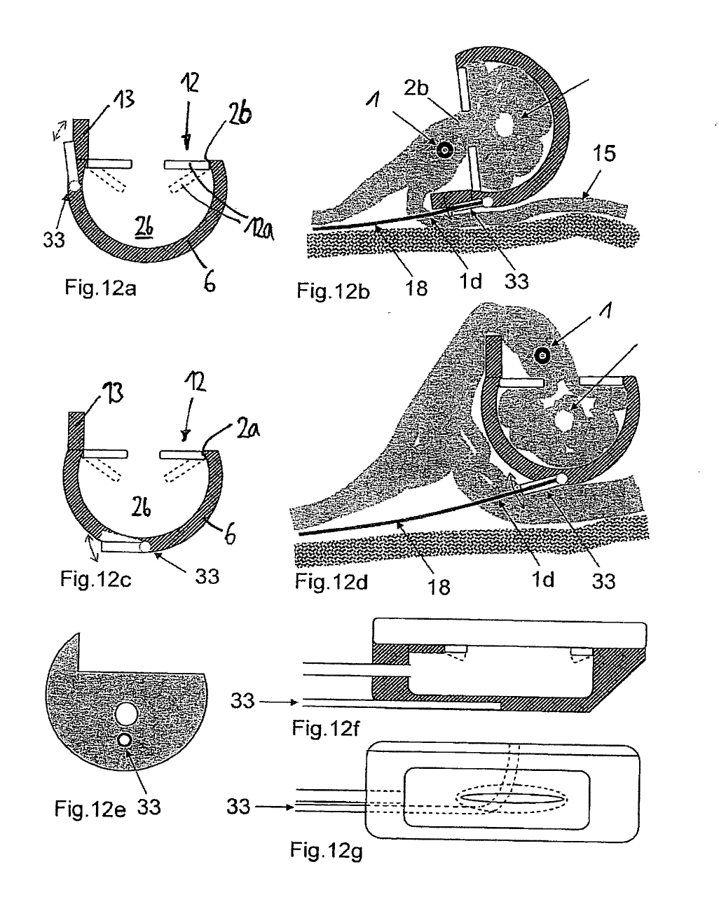

FIG. 12a a cross-section through a suction head of another embodiment of the device according to the present invention;

FIG. 12b the suction head of FIG. 12a in operation;

FIG. 12c a cross-section through a suction head of another further embodiment of the device according to the present invention;

FIG. 12d the suction head of FIG. 12c in operation;

FIG. 12e a cross-section through a tool holder for guiding the suction head of FIG. 12c;

FIG. 12f a longitudinal sectional drawing through the suction head of FIG. 12c, and

FIG. 12g a view from above of the suction head of FIG. 12c.

The device depicted in FIG. 1 serves for the fixation and manipulation of those tissues which are at least temporarily suitable for being fixed by suction. Hereby, tissues are understood to be, apart from organic tissues, e.g. tissues of animals and humans, also inorganic tissues, such as films or textiles.

The device serves, in particular, also for the fixation and manipulation of tissues, which surround sensitive organs, such as e.g. the pericardium in humans.

Thus, the device and the method to be carried out with the device are advantageous for those case constellations where several tissues are located above one another or where the outer tissue is located on a tissue or organ to be manipulated which is very sensitive to mechanical manipulation.

The invention is especially advantageous for accessing the interior of the pericardium, i.e. the pericardial sac; in particular, in those cases in which the pericardium is greatly thickened or in which the pericardium moves during the desired manipulation and an object located in the interior, for example, a further tissue or organ, likewise moves or is very sensitive to the desired manipulation.

In all cases, the device according to the present invention allows access to the pericardial sac after fixation and lifting of the pericardium through attachment to the device, for which reason the device can be referred to as “Attachlifter”.

The device according to the present invention comprises an oblong guiding body which comprises a connection on the lower end for a channel extending from there for the addition of a vacuum in an interior. The interior is located in a suction head at the distal end of the guiding body. The suction head comprises an opening in a side area which limits the interior. The opening in the side area of the guiding body is formed for the absorption of tissue. It comprises on its border means for the mechanical fixation of the tissue, wherein the means are stretching or clamping elements. These form, in the form of separated lips or in the form of a surrounding edge, a cross-section of the opening which is smaller than the cross-section of the interior limited by the opening. Thus, the stretching or clamping elements form an edge which protrudes into the plane of the opening of the interior, in such a way that undercuts or overhangs are formed.

The stretching or clamping elements (overhangs) are made of elastic material, in such a way that the opening which is limited by the clamping lips is initially expanded when the tissue is absorbed. However, as soon as an external force interacts with the tissue, by e.g., pulling on the tissue, the cross-section of the opening limited by the lips or the elastic border is reduced. The tissue is clamped by the lips or the edge respectively, even when the vacuum generation is interrupted. Thus, the means for the mechanical fixation of the tissue generate a retention force which prevailingly contributes to the functionality of the device.

The arrangement of the side opening with the stretching or clamping elements and the interior located beneath is chosen in such a way that areas comprising so-called undercuts, which additionally fixate the tissue once absorbed to the suctioning force of the vacuum, result for the tissue absorbed through the opening into the interior. The advantage consists of a mechanical adherence of the absorbed tissue when a force outwards is exerted. Hereby, clamping lips out of flexible material are advantageous, in order to seal the absorbed tissue even with an irregular surface in the suction opening.

During absorption of the tissue, the opening which is limited by the stretching or clamping elements is enlarged. When, however, a force outwards occurs, the opening is reduced, since the tissue tries to pull the elastic elements outwards. The tissue is held between the stretching or clamping elements and, moreover, sealed. This is then especially advantageous, when the device is rotated to lift the tissue from the organ located beneath.

It is also advantageous that the opening which is limited by the flexible stretching or clamping elements is variable. Such a suction head can thus be applied in types of clinical diseases with different tissue thicknesses.

Surprisingly, it has been found that considerably stronger attachment or fixation, respectively, is able to be achieved with the device according to the present invention, which also allows the attachment of very thick-walled and, thus, rigid tissue and, furthermore, reliable lifting of the tissue as well (also in the sense of separation from the underlying areas).

Further increase of the attachment effect is achievable when the clamping lips, after these were slanted inwards through absorbed tissue, are locked in this position (not pictured).

The stretching or clamping elements are also able to be actively moveable. They are able to form bodies which are, e.g., modifiable in their volume or in their extension in one or several directions in space. These are able be controlled, for instance through linkages, electric or fluid- or gas-hydraulic actuators or other elements able to be activated, or are able to be realized as electric or magnetic or fluid- or gas-hydraulic elements.

In a further embodiment (also not depicted), the device according to the present invention comprises an adjustment system, which is arranged through elements to determine the force or the degree of inclination on the moveable or actively moveable stretching or clamping elements and corresponding adjustment algorithms in such a way that the applied vacuum varies depending on the force of the absorbed tissue to the clamping lips or depending on the inclination of the clamping lips. Or the vacuum and the inclination of the clamping lips are varied in such a way that the tissue is absorbed continually or in steps deep in the side opening. In this, a modification of the clamping lips' inclination of up to 360° can also be provided.

The device is suitable for approaching the tissue with its side opening. After successful attachment, which e.g. can be reliably detected through the device for monitoring and display of the vacuum disclosed in DE-A1-103 37 813, an instrument guided in the guiding body for the manipulation or puncture of the tissue fixed to the suction head is able to be activated. In the case of puncture of the tissue, the area beneath the tissue is accessed, e.g. the interior of the pericardium for further manipulation, such as addition or removal of liquids or materials (e.g. electrodes or pharmaceutical substances).

In an advantageous embodiment, the device comprises, outside of the guiding body or of the interior of the hollow body, further means for the delivery of manipulation elements or optical elements or further elements known in medical technology or technology in the area of the attachment of the tissue to the device according to the present invention. This spatial separation causes the device to no longer require vacuum seals for the guidance of elements in the interior of the guiding body. Furthermore, the advantage that the area of attachment is spatially separated from the area of manipulation or optical detection results. This is advantageous particularly for very thick-walled tissues, as these often fill the interior of the hollow body completely with the wall of the tissue and, thus, puncture of the tissue in the interior does not enable accessibility to the areas located beneath the wall.

This circumstance is encountered particularly in the medical application of the device, where, for instance in the area of minimally invasive surgery, the interior is not able to be realized with any desired volume.

FIG. 1 depicts the fundamental components of the device according to the present invention. It comprises a guiding body 5 with a suction head 6. The latter comprises an interior 2a which is limited by a side opening 2b. Within the guiding body, a channel 7 is realized which ends with an opening 8 in the interior 2a. The channel 7 serves as means for the introduction of a vacuum to the interior 2a. For this purpose, furthermore, a vacuum source 4 is connected to the channel 7.

Alongside and parallel to the channel 7, a guiding channel 3a is realized for the penetrating body 1. The latter is able to be a needle, scissors, a blade, forceps, a wire or an electrode. It is activated and/or guided by an optional guiding device 3a.

The device comprises optionally an attachment detection device 9, as described in DE-A1-103 37 813. This is able to comprise a pressure sensor, as well as display device 11 in order to display whether the required suction pressure has been achieved in the interior 2a. In this way, the user of the device is able to detect whether sufficient fixation has been achieved through the vacuum. The pressure sensor 9 is also able to be arranged within the suction head 6 or in the area beneath in the device.

In order to allow for puncture or other manipulation, e.g. radiation, the penetrating body 1 is slid forwards and backwards in the direction of its longitudinal axis and/or rotated within the guidance 3a by means of a guiding device 3b. Hereby, it is important that the penetrating body 1 does not lie within the vacuum channel 7, but is arranged within a separated channel 3a, so that a spatial distance is generated between the penetrating body 1 and the interior 2a of the suction head 6. Furthermore, it is not necessary to provide the guidance 3a of the penetrating body 1 with vacuum seals, causing positive effects on the production costs and the reliability of the device.

The penetrating body 1 is inserted into the device from the proximal end. A retaining device is provided which holds the penetrating body in a position “before the puncture” of a tissue or material. The retaining device is able to be realized in the form of a simple mechanical device, e.g. in the form of a clamp. After the reliable detection of the attachment of the tissue or organ, the penetrating body 1 can be inserted in this tissue or organ. The tool holder for the guidance of the hollow or guiding body or only of the suction head is not depicted.

Preferentially, a continuously operating suction pump (without readjustment of the pressure in the case of pressure changes) or a vacuum source continually working with constant performance is used as a vacuum source 4, in order to be able to detect eventual leaks in the attachment. The detection of the attachment is reached through prior gauging or calibration by means of pressure measurement with closed suction head.

FIG. 2 shows a sectional drawing of an advantageous embodiment, approximately in the longitudinal plane of the guiding or hollow body 5 of the device, in the area of the suction head 6. The interior area 2a of the suction head 6 with the overhanging stretching or clamping elements 12, which form undercuts in relation to the edge of the opening 2b, is clearly recognizable. The stretching or clamping elements 12 are advantageously arranged as moveable clamping lips 12a which are able to lead to a bigger or smaller cross-section of the opening 12c. It is also recognizable that the opening 8 to the channel 7 conducting the vacuum is located considerably spaced from the left edge of the upper opening 2b. This embodiment ensures the advantage that even a relatively large area of tissue is able to be absorbed before the effect of suction decreases due to covering of the vacuum opening 8.

FIG. 3 shows a suction head in a view from above in which the stretching or clamping elements 12 form a surrounding edge 12b. Here as well, overhangs with undercuts are formed which fixate the tissue 15 within the suction head 6.

FIG. 4 shows the sectional drawing approximately perpendicular to the longitudinal plane of the suction head from the FIG. 2,3. This suction head features a molding 13) in the area of the opening 2a, 2b), here in the form of a wall. Through this molding, among others, the friction of the tissue on the device and, thus, the fixating effect (e.g. during rotation) is increased. Above all, a barrier between the manipulation device and tissue is formed (thus also called a protective lip).

FIG. 5 shows the sectional drawing approximately perpendicular to the longitudinal plane of a tool holder 14 for guidance of the hollow or guiding body 5 or, in case of a device with multiple parts, the suction head 6 alone as well, and namely with a molding 13, such as is provided on the suction head as well, so that the user/operator always receives direct feedback concerning the position of the suction head.

FIG. 6 a, b show a suction head according to FIG. 4 in action and, namely in 6a, after suction of tissue 15, which is able to be lifted from the movable tissue/organ 15a located beneath. FIG. 6a shows the step b) of the method according to the present invention and, FIG. 6b, the state after rotation of approx. 90° to the right (after step c2) of the method according to the present invention. Hereby, an area of manipulation 16 results in the area of lifting 17. A further advantageous effect becomes visible, consisting in the fact that the molding 13 forms a barrier between the manipulation area 16 and the tissue/organ 15a. Furthermore, it is shown in FIG. 6c,d that also in the case of thick-walled tissue, which is able to completely fill the suction head, an area of lifting 17 is created for manipulation in the manipulation area 16.

FIG. 7 a, b, c show a further advantageous embodiment of the device with a cutting device, loop or other manipulation devices located on the suction head. In a), the tissue is absorbed into the suction head; in b), tissue manipulation takes place in the manipulation area 16) after rotation to the right (e.g. penetration of a needle 1 with guiding wire 18 located in the needle). This advantageous embodiment of the penetrating body is depicted in the cross-section as a point in the manipulation area—for clarification, the needle is also depicted in the longitudinal sectional drawing. In c), the tissue located in the suction head is separated with the help of a cutting device (e.g. blade 20, loop) moving in a guidance 19). In c), the further procedure when accessing through the pericardial tissue into the underlying pericardial sac is also schematically depicted. After the needle has punctured the pericardium, the guiding wire is pushed forwards into the pericardial sac. A dilator 22) is slid along the guide wire in the direction of the opening 21a) created by the needle. Through expanding the opening, the narrow connection/bridge pierces to the opening created by the cutting device 21b). With this method, a large opening in the pericardium (with guiding wire located in the opening and slid forwards into the pericardial sac) required for subsequent manipulations is created. An advantageous method resulting from this is the epicardial implantation of a screw electrode 23) (e.g. for pacemakers schematically depicted).

FIG. 8 a, b, c show a sectional drawing in the longitudinal plane, a view from above and a sectional drawing approximately perpendicular to the longitudinal plane of a possible embodiment, as described in FIG. 7.

FIG. 9 shows an advantageous embodiment with possible dimensioning in mm for access into the pericardial sac, with a needle as penetrating body 1) and assigned guiding device 3) and, optionally, a cutting device in the form of a movable blade 20 with assigned guiding device 20a and a guidance for an endoscope 24). a) depicts a sectional drawing approximately in the longitudinal plane of the device, and b) a sectional drawing approximately perpendicular to the longitudinal plane at the level of the tool holder outside of the guiding device 3 (the blade guidance is referred to with 20b). In a), the guidances for needle and blade, which prevent injury of the tissue before the actual tissue manipulation, are not depicted.

FIG. 10 shows a flexible embodiment of the hollow or guiding body with internal, but separated and thus spaced, channels for vacuum and guidance of the possible manipulating or penetrating bodies. Advantageous manipulation devices are: in a) and b) electrodes (the fixation of tissue through suction makes the fastening of a screw electrode into the tissue/organ easier), in c) a cutting device and in d) a penetrating body in the form of a needle. Advantageous locations of manipulation are: in a) outside of the suction head, and in b)-d) inside of the suction head. In b)-d), the manipulation devices move in the vacuum channel. In these embodiments, no further lifting or raising of the tissue, e.g. rotation from FIG. 6), in the sense of the method c2) according to the present invention is necessary. The suction heads comprise overhangs (clamping lips) with areas featuring undercuts 12a). In b), the screw electrode is guided through the suction head and, thus, there is no overhang in this area. An optical fiber (endoscope) 24) is advantageous for the observation of the tissue-manipulating bodies 23). In FIG. 10, a tissue/organ is referred to with 15a and a tissue able to be raised/moved with 15.

FIG. 11 a, b, c schematically show a method with use of the method according to the present invention and a device according to the present invention, here with a flexible guiding or hollow body, during minimally invasive manipulations in the pericardial sac. Included within are a) view from above, b) longitudinal sectional drawing, c) cross-section. The method is characterized by the steps: the suction head is placed beneath the xiphoid process 27) on the pericardium 30) after access into the thoracic cavity, the pericardium is punctured, and a guiding wire is slid forwards into the underlying pericardial sac 31). Subsequently, further manipulations are able to be performed. An advantageous manipulation is the implantation of a screw electrode 23) (e.g. for pacemakers depicted schematically at the tip of the left heart chamber). In the case of implantation of electrodes on the surface of the heart (epicardium) 32), no restriction of lung function occurs. In the advantageous embodiment of the application of the device to the heart, the movable tissue to be lifted (15) from FIGS. 6 and 7 corresponds to the pericardium 30). The underlying tissue/organ 15a) corresponds to the epicardium 32) and heart muscle.

In the embodiment of FIG. 12, suction takes place as already described with subsequent rotation of the device of approx. 90° followed by a forward movement of the needle. As a strongly adipose pericardium is concerned in this case, the tissue fold consists mainly of fat tissue and the needle does not reach the pericardial sac. Thus, the suction head with the tissue fixed by the needle is rotated a further 90° degrees. Hereby, the tissue is “clamped” on the suction head by the needle pierced in the tissue and the force of the pericardium grown on several sites to organs. This clamping preferentially requires the penetration of a needle (or, generally, of another fixation device). In this case, the vacuum-mediated suction is insufficient and the tissue would detach again from the suction head in the case of a rotation of approx. 180°. Subsequently, preferentially a wire, dilator, needle, etc. is moved forward into the clamped tissue perpendicularly to the clamped tissue. If the wire, when doing so, is not moved in a manner parallel to the pericardium surface, but inclined as compared to the heart surface, it punctures the pericardium at any thickness of the tissue. With this device, it is thus possible to perforate a pericardium of variating thickness. When the dilator reaches the other side of the pericardium, injuries of the frequently narrowly spaced epicardium do not occur, as the preferentially used flexible wire (preferentially a flexible guide wire with spiral arrangement) does not have any more mechanical guidance in the pericardial sac which predefines a directed movement. Thus, the flexible wire is not forced to penetrate the epicardium but is able to simply move forwards in the pericardial sac. The forward movement of the flexible wire is also conducted in very small steps and the operator feels when he overcomes the resistance during puncturing the outer connective tissue-side followed by puncturing of the inner connective tissue-side. Should the wire keep moving in the fat tissue, this is detected, and the wire is moved back and moved forward again after a rotation. During this procedure, the pericardium remains closely clamped to the device. This embodiment 1 requires that the pericardium is tightly stretched and, thus, is clamped to the device after rotation of 180°.

In the case of a less tightly stretched tissue (widened, e.g., as consequence of a preceding pericardial effusion), the suction head with the tissue fixed by the needle is not only rotated by 180°, but preferentially by approx. 360° degrees. Hereby, the tissue is strongly clamped to the suction head through winding. The method requires that sufficient tissue (2*2*pi*radius of the suction head) is available for winding.

A further (not depicted) embodiment is applied to a not tightly stretched tissue, wherein in this case a rotation of only 180° is conducted. In order to achieve sufficient clamping, a head piece with 2 recesses or suction heads staggered by 180° to one another is used. First, the first suction head with suctioned tissue is rotated 90° to the left and the needle punctures the thickened pericardium. The head is subsequently further rotated by 90° and the second head, now located on the pericardium, is suctioned, rotated 90° to the right and the second needle punctures the thickened tissue. The head is then turned approx. 90° to the left (in total)180°. Now the tissue is “clamped” to the suction head on both sides of the suction head and is not able to give way to the subsequent manipulation which takes place perpendicularly to the clamping.

FIG. 12 a and b show a suction head which comprises a further guidance 33 for a further penetrating body 18. This guidance is able to be rotated as compared to the suction head 6 in such a way that a penetrating body 18 guided in its interior, e.g. a wire, is able to be introduced perpendicularly to the suction head 6 into the tissue 15.

In FIG. 12a, the guidance 33 is realized underneath the molding 13 on the side of the suction head 6. In FIG. 12c, the guidance 33 is realized opposite the opening 2b.

The invention is not limited to one of the previously described embodiments, but is suitable for being modified in all kinds of ways. One recognizes, however, that a device for puncturing or/and other manipulations of organic or inorganic tissue or underlying areas, comprises at least one oblong hollow or guiding body 5), e.g. in the form of a rigid or flexible tube or rod. Furthermore, the device comprises means for guidance of at least one penetrating body 1), as well as means for the addition, e.g. in the form of a channel 7), of a vacuum into an interior of the body 5) which comprises at least one side opening 2). In order to improve the fixation of the tissue, the opening in the side area of the hollow body for the formation of a suction head 6) is arranged or suitable for being arranged in such a way that the cross-section of the opening, for instance through a surrounding edge or individual pieces protruding into the plane of the opening or in another form, is realized smaller than the cross-section of the interior of the device located below, parallel to the plane of the opening.

All of the characteristics and advantages originating from the claims, description and figures, including constructive details, spatial arrangements and processing steps, are suitable for being essential to the invention, both in themselves and in the most different combinations.

### LIST OF REFERENCE NUMERALS

1 penetrating body
2a interior
2b side opening
3a guidance
3b guiding device
4 vacuum source
5 guiding body
6 suction head
7 vacuum channel
8 vacuum opening
9 attachment detection device
10 pressure sensor
11 display device
12 mechanical fixation means
12a elastic lip
12b elastic edge
12c cross-section of the opening
13 molding, ground/protective lip
14 tool holder
15 tissue
16 manipulation area
17 area of lifting
18 guide wire
19 guidance for cutting device
20 blade or loop
21 tissue opening
22 dilator
23 tissue-manipulating body
24 endoscope
25 ribs
26 sternum
27 xiphoid process
28 lungs
29 heart chambers (a, right; b, left)
30 pericardium
31 percardial cavity (pericardial sac)
32 epicardium of the heart muscle

1. Device for the fixation and/or manipulation of tissue (15) and/or underlying areas (16), with at least one body (1) which penetrates the tissue (15) and/or the underlying areas (16) and one guiding body (5) for the penetrating body, wherein the guiding body (5)
comprises means (3a) for the guidance of the penetrating body (1),
has or forms a suction head (6) at its end for the tissue (15),
comprises within the suction head (6) an interior (2a) with at least one opening (2b) arranged in the side area of the guiding body (5) to draw in the tissue (15) to be punctured and/or manipulated, and
comprises means (7) for the introduction of a vacuum into the interior (2a),
wherein
at least one means (12) for mechanical fixation and/or attachment of the tissue (15) to the suction head (6) is provided in the area of the opening (2b) of the suction head (6).

2. Device according to claim 1, wherein the at least one means (12) for the mechanical fixation and/or attachment of the tissue (15) to the suction head (6) is a stretching or clamping element.

3. Device according to claim 2, wherein each elastic stretching or clamping element (12) is able to form an elastic lip 812a), spring or tongue which protrudes at least partially into the opening (2b).

4. Device according to claim 2, wherein the elastic stretching or clamping element (12) forms a surrounding elastic edge for the opening (2b).

5. Device according to claim 2, wherein the cross-section of the opening (12c) limited by the elastic stretching or clamping element (12) is smaller than the cross-section of the interior (2a) limited by the opening (2b).

6. Device according to claim 2, wherein the elastic stretching or clamping elements (12) are able to be actively moveable or modifiable.

7. Device according to claim 2, wherein the elastic stretching or clamping elements (12) are moveable mechanically, electrically, magnetically, hydraulically or pneumatically.

8. Device according to claim 1, wherein the at least one means (12) for the mechanical fixation and/or attachment of the tissue (15) to the suction head (6) is the penetrating body (1).

9. Device according to claim 1, wherein the penetrating body (1) is slide guided outside the suction head (6).

10. Device according to claim 1, wherein the means (3a) for guidance of the penetrating body (1) are realized separated from the means (7) for the introduction of a vacuum into the interior (2a).

11. Device according to claim 1, wherein the penetrating body (1) is a needle, an electrode, a wire, a loop, a cutting device or something similar.

12. Device according to claim 1, wherein the means (7) for the introduction of a vacuum into the interior (2a) is a vacuum channel which ends in the interior (2a) with a vacuum opening (8).

13. Device according to claim 12, wherein the vacuum opening (8) is realized at a distance (a) from the edge of the opening (2b).

14. Device according to claim 1, wherein at least one further guidance (33) for another penetrating body (18) is provided in the suction head (6).

15. Device according to claim 14, wherein the further guidance (33) is realized in a rotatable manner in relation to the suction head (6).

16. Device according to claim 14, wherein the further penetrating body (18) is a guide wire.

17. Device according to claim 1, wherein the suction head (6) comprises at least one molding (13).

18. Device according to claim 1, wherein the molding (13) is realized in the border area of the suction head (6).

19. Device according to claim 1, wherein the suction head (6) in the area of the side opening (2b) is realized in a flattened manner.

20. Device according to one of the claims 1 to 19 claim 1, wherein at the suction head (6) a cutting device (20) for the tissue (15) is realized.

21. Method for the fixation and/or manipulation of tissue (15) and/or underlying areas (16) with a device according to claim 1, characterized by the following steps:
a) approach of the device according to the present invention to the tissue (15) to be fixed;
b) suction of the tissue (15) through the side opening (2b) of the device until attachment of the tissue (15) and mechanical fixation of the tissue (15);
c) further lifting or raising of the tissue (15) from the position of the tissue before step b);
d) approach of the at least one penetrating body (1) to the tissue in the spaced area between the position of the tissue before step b) and the tissue area in the interior of the device spaced the furthest therefrom.

22. Method according to claim 21, wherein further lifting or raising in step c) occurs through rotation of the oblong hollow body around its longitudinal axis and, thus, also of the tissue attached to the hollow body in the area of the side opening.

23. Method according to claim 21, wherein, in step d), the approach of the penetrating body (1) to the tissue (15) in the spaced area between the position of the tissue before step b) and the tissue area in the interior of the device spaced the furthest therefrom occurs outside of the area of the interior of the hollow body conducting the vacuum.

24. Suction head for the absorption of tissue, comprising a body with an interior, which is realized for the addition of a vacuum to the interior, and wherein the body comprises an opening on the surface, which is connected to the interior for the addition of a vacuum to the opening, wherein the opening is molded or arranged to be molded in such a way that the cross-section of the opening, for instance through a surrounding edge or individual pieces protruding into the plane of the opening or in another form, is realized smaller than the cross-section of the interior of the suction head located below, parallel to the plane of the opening.

25. Device according to claim 1, comprising a suction head comprising a body with an interior, which is realized for the addition of a vacuum to the interior, and wherein the body comprises an opening on the surface, which is connected to the interior for the addition of a vacuum to the opening, wherein the opening is molded or arranged to be molded in such a way that the cross-section of the opening, for instance through a surrounding edge or individual pieces protruding into the plane of the opening or in another form, is realized smaller than the cross-section of the interior of the suction head located below, parallel to the plane of the opening.

26. In a method of manipulating and/or puncturing organic or inorganic tissues, the improvement comprising using the device of claim 1.
